(19) United States
(12) Patent Application Publication
(10) Pub. No.: US 2022/0021031 A1
(43) Pub. Date: Jan. 20, 2022

(54) ELECTROLYTE ADDITIVE, ELECTROLYTE, LITHIUM ION SECONDARY BATTERY CONTAINING THE SAME AND USE THEREOF

(71) Applicant: Murata Manufacturing Co., Ltd., Nagaokakyo-shi (JP)

(72) Inventors: Manli XUE, Shanghai (CN); Haoyue ZHONG, Shanghai (CN); Cheng ZHU, Shanghai (CN); Hao ZHANG, Shanghai (CN)

(21) Appl. No.: 17/366,232

(22) Filed: Jul. 2, 2021

(30) Foreign Application Priority Data
Jul. 7, 2020 (CN) 202010646754.X

(51) Int. Cl. H01M 10/0567; H01M 10/0525; C07D 471/08; C07D 211/94

(52) U.S. Cl. CPC H01M 10/0567; H01M 10/0525; H01M 2300/004; C07D 211/94; C07D 471/08

(57) ABSTRACT

An electrolyte additive includes any one or more compounds in a group consisting of compounds in Formula (1) and Formula (2) below, wherein R₁ to R₄ are respectively independently selected from a group consisting of H, C₁₋₆ alkyl and halogen, R₅ and R₆ are respectively independently selected from a group consisting of H, C₁₋₆ alkyl and aromatic hydrocarbon, R7 is independently selected from a group consisting of H, C₁₋₆ alkyl, C₁₋₆ alkoxy, a nitrile group, an ester group, an amide group, an amino group, and a maleimide group, optionally, R₅ and R₆ are respectively combined with R₇ or together with R₇ and atoms to which they are connected to form a 6-14-membered ring structure.

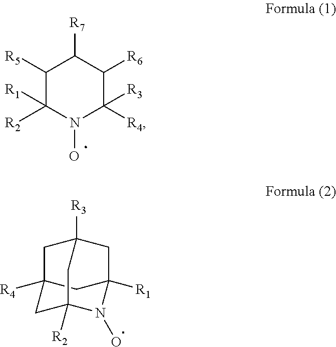

### CROSS REFERENCE TO RELATED APPLICATIONS

This application claims the benefit of Chinese Patent Application No. 202010646754.X, filed on Jul. 7, 2020, and titled “Electrolyte Additive, Electrolyte, Lithium Ion Secondary Battery Containing the Same and Use thereof”, the entire contents of which are incorporated by reference herein.

### BACKGROUND OF THE INVENTION

### 1. Field of the Invention

The present disclosure relates to the field of lithium ion secondary batteries, and in particular, to an electrolyte additive, electrolyte, lithium ion secondary battery including the same and use thereof.

### 2. Description of the Related Art

In recent years, along with continuous development of an electronic technology, the requirements for people to use a battery device for supporting energy supply of an electronic device are also continuously increased. Nowadays, batteries capable of storing a high amount of electricity and outputting high power are needed. Traditional lead-acid batteries, nickel-metal hydride batteries and the like may not meet the requirements of mobile equipment, such as a smart phone, and a new-type electronic product of fixed equipment, such as a power storage system. Therefore, a lithium battery has attracted extensive attention. During the development process of the lithium battery, capacity and performance thereof have been more effectively improved.

At present, an electrolyte of a widely used lithium ion secondary battery is mainly composed of a mixture solution including lithium hexafluorophosphate as a conductive lithium salt and including cyclic carbonate and chain carbonate as a main mixed solvent. However, the above-described electrolyte still has many disadvantages. For example, during the first charging and discharging processes of the lithium battery, a negative electrode material may react with the electrolyte to form a passivation layer (namely, a solid electrolyte interface membrane, referred to as an SEI film) covering the surface of the negative electrode material. The SEI film has the characteristics of a solid electrolyte, and it is an insulator of the electron, but a good conductor of lithium ions (Li⁺). Li ions may be freely intercalated and de-intercalated through the SEI film. The stability of the SEI film is critical to the cycle performance of the battery. The stable SEI film may significantly improve the performance of the battery. On the contrary, if the SEI film is unstable, the SEI film may continue to grow during the charging and discharging processes, thereby the polarization and internal resistance of the battery are increased, and the cycle performance of the battery is further degraded. The use of an electrolyte film-forming additive is a simple and efficient method to improve the battery cycle stability. At present, a commonly used method is to add a small amount of an additive to the electrolyte. The electrolyte additive may react with the electrode material in preference to the solvent and form the stable SEI film on the surface of the negative electrode. As a result, the co-intercalation of solvent molecules and the damage of the negative material due to the co-intercalation of the solvent molecules are inhibited. Commonly used additives include fluoroethylene carbonate (FEC), vinylene carbonate (VC) and so on.

In the prior art, the most commonly used negative electrode film-forming additive in the lithium ion secondary battery is fluoroethylene carbonate (FEC). The FEC has a lower energy in a lowest unoccupied molecular orbital (LUMO), and is easy to be reduced. It is generally considered as a good negative electrode film-forming additive. A relative dielectric constant of the FEC is higher than that of ethylene carbonate (EC), the melting point is lower than that of the EC, and fluorine atoms are included. Thus, it is beneficial to the infiltration of the electrode and separator, and it is conducive to improving the capacity and low-temperature performance of the battery. Because a fluorine-containing structure has better oxidation resistance, FEC is often used in high-voltage electrolytes, and its advantageous effect is usually proportional to its dosage. However, the large volume use of FEC may bring higher viscosity and higher cost, and therefore other properties of the lithium ion secondary battery are degraded. In addition, under a high temperature condition, FEC is easily decomposed to generate carbon dioxide, resulting in serious aerogenesis and a risk of battery explosion. Therefore, it is necessary to control the dosage of FEC.

Therefore, in order to solve the problems as mentioned above, it is still necessary to develop an electrolyte additive that may effectively form the SEI film, reduce the dosage of FEC, and ensure the electrical performance of the lithium ion secondary battery.

### SUMMARY OF THE INVENTION

Example preferred embodiments of the present disclosure provide electrolyte additives, electrolytes including the electrolyte additives, lithium ion secondary batteries including the electrolytes, and uses of the electrolyte additives, so as to solve problems that the electrical performance of the lithium ion secondary battery is poor and the dosage of a film-forming additive is large in the prior art.

According to one aspect of an example preferred embodiment of the present disclosure, an electrolyte additive includes any one or more compound(s) selected from a group consisting of compounds as shown in Formula (1) and Formula (2) below:

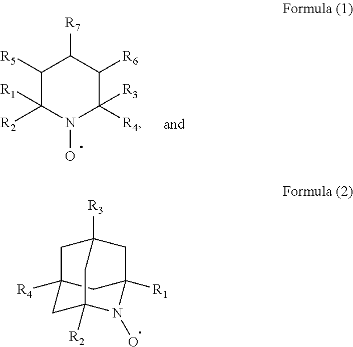

wherein, R₁ to R₄ are respectively independently selected from a group consisting of H, C₁₋₆ alkyl and halogen, R₅ and R₆ are respectively independently selected from a group consisting of H, C₁₋₆ alkyl and aromatic hydrocarbon, R₇ is independently selected from a group consisting of H, C₁₋₆ alkyl, C₁₋₆ alkoxy, a nitrile group, an ester group, an amide group, an amino group, and a maleimide group, and optionally, R₅ and R₆ are respectively combined with R₇ or R₅ and R₆ are combined together with R₇ and atoms to which they are connected to form a 6-14-membered ring structure.

Further, in the above electrolyte additive, in the compound shown in Formula (1), wherein R₅ and R₆ are H respectively, and optionally, R₅ and R₆ are respectively combined with R₇ or R₅ and R₆ are combined together with R₇ and the atoms to which they are connected to form the 6-14-membered ring structure.

Further, in the above electrolyte additive, in the compound shown in Formula (2), wherein R₁ to R₄ are respectively independently selected from a group consisting of H, C₁₋₃ alkyl, and F.

Further, in the above electrolyte additive, wherein the compound shown in Formula (1) is selected from the following compounds:

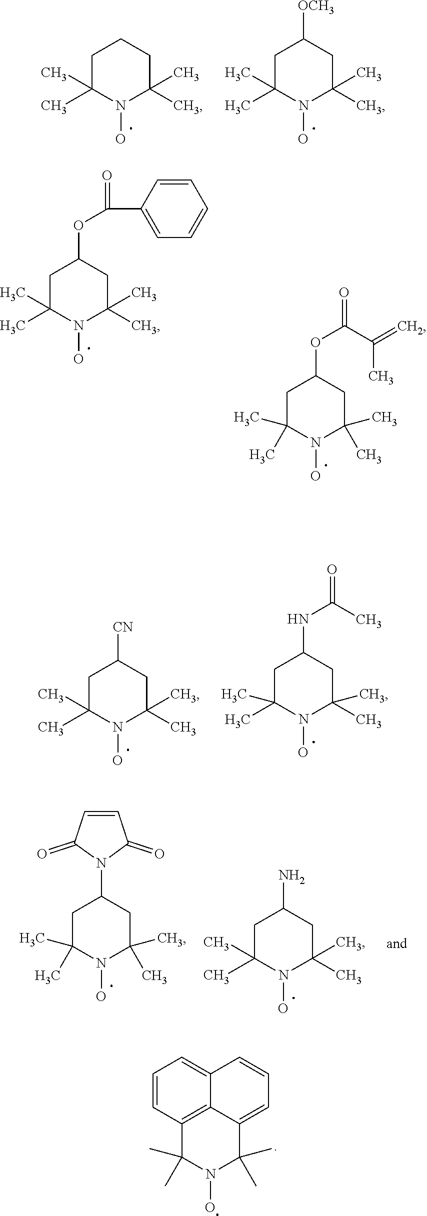

Further, in the above electrolyte additive, the compound shown in Formula (2) is selected from the following compounds:

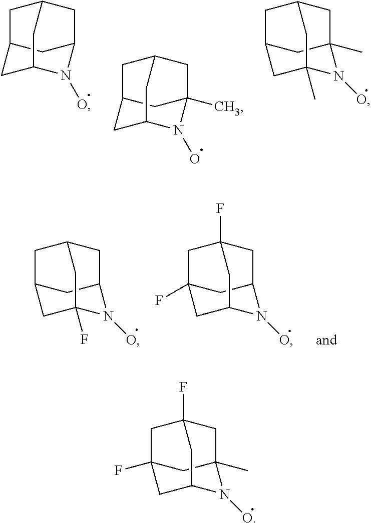

According to another example preferred embodiment of the present disclosure, an electrolyte includes an organic solvent, a lithium salt, a film-forming additive, and the electrolyte additive as mentioned above.

Further, in the above electrolyte, an amount of the electrolyte additive ranges from about 0.01 parts by weight to about 1 part by weight, based on 100 parts by weight of a total weight of the organic solvent, the lithium salt, and the film-forming additive.

Further, in the above electrolyte, the lithium salt is selected from a group consisting of LiCl, LiBr, LiPF₆, LiBF₄, LiAsF₆, LiClO₄, LiB(C₆H₅)₄, LiCH₃SO₃, LiCF₃SO₃, LiN(SO₂CF₃)₂, LiN(SO₂F)₂, LiC(SO₂CF₃)₃, LiAlCl₄, LiSiF₆, or any combinations thereof.

According to another example preferred embodiment of the present disclosure, a lithium ion secondary battery includes a positive electrode, a negative electrode, a separator, and the electrolyte as mentioned above.

According to another example preferred embodiment of the present disclosure, a method of using the electrolyte additive as mentioned above in preparation of a lithium ion secondary battery is provided.

By using the electrolyte additive, the electrolyte, the lithium ion secondary battery including the same and the use thereof of the present disclosure, technical effects of improving the cycle stability of the lithium ion secondary battery, reducing resistance of battery after a charging and discharging cycle, and reducing a usage amount of the film-forming additive are achieved.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 shows cyclic voltammetry curves of first cycles of batteries of Example 20 and Comparative Example 12.

FIG. 2 shows the cyclic voltammetry curves of the batteries of Example 20 and Comparative Example 12.

FIG. 3 shows EIS measurements of the batteries of Example 20 and Comparative Example 12 under full power.

FIG. 4 shows capacity versus cycle number of batteries of Example 23, Example 24, and Comparative Example 14.

### DETAILED DESCRIPTION OF THE PREFERRED EMBODIMENTS

It is noted that example preferred embodiments in the present disclosure and features in the example preferred embodiments may be mutually combined with each other without departing from the present disclosure. The example preferred embodiments of the present disclosure are described in detail below in combination with the example preferred embodiments. The following example preferred embodiments are only exemplary, and do not constitute limitations on a scope of protection of the present disclosure.

As described in the background, fluoroethylene carbonate (FEC) is generally used as a negative electrode film-forming additive in a lithium ion secondary battery in the prior art. However, when the fluoroethylene carbonate is used, problems such as increased viscosity of electrode, increased cost, gas generation, and degradation of the cycle performance of the lithium ion secondary battery may occur. In view of the problems with the prior art, an example preferred embodiment of the present disclosure provides an electrolyte additive including any one or more compound(s) selected from a group consisting of compounds as shown in Formula (1) and Formula (2) below:

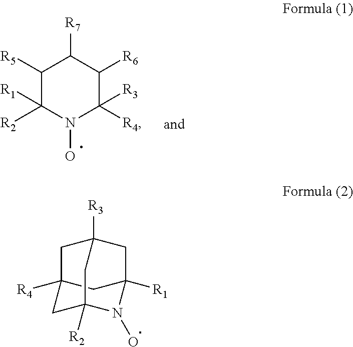

wherein R₁ to R₄ are respectively independently selected from a group consisting of H, C₁₋₆ alkyl and halogen, R₅ and R₆ are respectively independently selected from a group consisting of H, C₁₋₆ alkyl and aromatic hydrocarbon, R₇ is independently selected from a group consisting of H, C₁₋₆ alkyl, C₁₋₆ alkoxy, a nitrile group, an ester group, an amide group, an amino group, and a maleimide group, and optionally, R₅ and R₆ are respectively combined with R₇ or R₅ and R₆ are combined together with R₇ and atoms to which they are connected to form a 6-14-membered ring structure.

After a larger number of experiments were performed, the inventors of example preferred embodiments of the present disclosure surprisingly discovered that: when adding a compound containing N—O. free radical into electrolyte, the compound containing the N—O. free radical may effectively improve a decomposition potential of fluoroethylene carbonate (FEC), since the oxygen atom has a lone electron. Furthermore, the FEC decomposed at a high potential level is more conducive to form a stable SEI film.

Specifically, a compound containing a stable N—O. free radical (organic N—O free radical) according to an example preferred embodiment of the present disclosure is often used as a catalyst in organic synthetic reaction in the prior art. After a large amount of research and experiments were performed, the inventors of present disclosure surprisingly discovered that in the case that the compound containing the N—O. free radical of an example preferred embodiment the present disclosure and the fluoroethylene carbonate are simultaneously comprised in the electrolyte, the compound containing the N—O. free radical may perform the following reversible reaction:

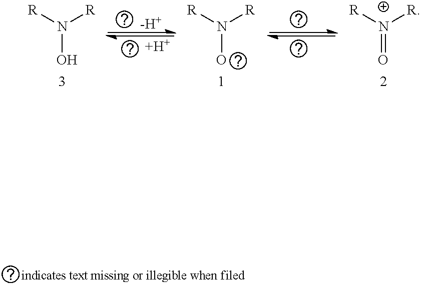

In an example preferred embodiment of the present disclosure, an exemplary compound containing N—O. free radical may carry out the following reactions in the electrolyte of the lithium ion secondary battery containing the FEC:

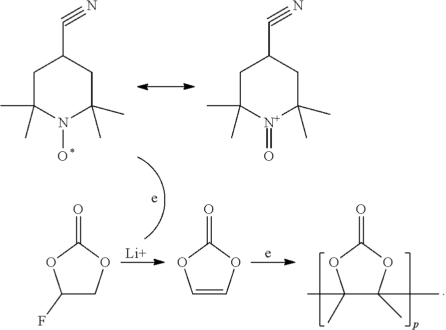

The compound containing the N—O. free radical according to an example preferred embodiment of the present disclosure may provide electrons to the FEC, thereby the FEC is promoted to carry out a reductive decomposition reaction at the high potential, so that the decomposition of the FEC precedes the decomposition reaction of the electrolyte of the lithium ion secondary battery. As a result, it is beneficial to form a more stable SEI film, and the resistance of the lithium ion secondary battery during the first film-forming process is significantly reduced. In the case of using the compound containing the N—O. free radical according to an example preferred embodiment of the present disclosure and the FEC simultaneously, because the compound containing the N—O. free radical according to an example preferred embodiment of the present disclosure promotes the decomposition of the FEC and film formation, the simultaneous use of the compound containing the N—O. free radical according to an example preferred embodiment of the present disclosure and FEC may reduce the usage amount of the FEC, reduce the internal resistance of the lithium secondary battery and improve the high temperature performance and/or rate performance of the secondary battery, compared with the case of using the FEC only.

In some example preferred embodiments of the present disclosure, the electrolyte additive may include one of the following substituted piperidine-N-oxide based compound or any combination thereof: 2-methyl-N-oxopiperidine (also called as 2-methyl-piperidine-N-oxide), 2-ethyl-N-oxopiperidine, 2-propyl-N-oxopiperidine, 2-butyl-N-oxopiperidine, 2-pentyl-N-oxopiperidine, 2-hexyl-N-oxopiperidine, 2,3-dimethyl-N-oxopiperidine, 2,4-dimethyl-N-oxopiperidine, 2,5-dimethyl-N-oxopiperidine, 2,6-dimethyl-N-oxopiperidine, 3,4-dimethyl-N-oxopiperidine, 3,5-dimethyl-N-oxopiperidine, 3,6-dimethyl-N-oxopiperidine, 2,3,4-trimethyl-N-oxopiperidine, 2,3,5-trimethyl-N-oxopiperidine, 2,3,6-trimethyl-N-oxopiperidine, 3,4,5-trimethyl-N-oxopiperidine, 3,4,6-trimethyl-N-oxopiperidine, 2.3.4.5-tetramethyl-N-oxopiperidine, 2,3,5,6-tetramethyl-N-oxopiperidine, 2,3,4,5,6-pentamethyl-N-oxopiperidine, 2,2,3-trimethyl-N-oxopiperidine, 2,2,4-trimethyl-N-oxopiperidine, 2.2.5-trimethyl-N-oxopiperidine, 2,2,6-trimethyl-N-oxopiperidine, 2,2,3,4-tetramethyl-N-oxopiperidine, 2,2,3,5-tetramethyl-N-oxopiperidine, 2,2,3,6-tetramethyl-N-oxopiperidine, 2,2,3,3-tetramethyl-N-oxopiperidine, 2,2,3,4,5-pentamethyl-N-oxopiperidine, 2,2,3,4,6-pentamethyl-N-oxopiperidine, 2,2,3,4,5,6-hexamethyl-N-oxopiperidine, 2,2,6,6-tetramethyl-N-oxopiperidine, 2,2,3,6,6-pentamethyl-N-oxopiperidine, 2,2,4,6,6-pentamethyl-N-oxopiperidine, 2.2.3.4.6.6-hexamethyl-N-oxopiperidine, 2,2,3,5,6,6-hexamethyl-N-oxopiperidine, 2,2,3,4,5,6,6-heptamethyl-N-oxopiperidine, 2,3-diethyl-N-oxopiperidine, 2,4-diethyl-N-oxopiperidine, 2,5-diethyl-N-oxopiperidine, 2,6-diethyl-N-oxopiperidine, 3,4-diethyl-N-oxopiperidine, 3,5-diethyl-N-oxopiperidine, 3,6-diethyl-N-oxopiperidine, 2,3-dipropyl-N-oxopiperidine, 2,4-dipropyl-N-oxopiperidine, 2,5-dipropyl-N-oxopiperidine, 2,6-dipropyl-N-oxopiperidine, 3,4-dipropyl-N-oxopiperidine, 3,5-dipropyl-N-oxopiperidine, 3,6-dipropyl-N-oxopiperidine, 2,6-dimethyl-4-methoxy-N-oxopiperidine, 2,6-dimethyl-4-ethoxy-N-oxopiperidine, 2,6-dimethyl-4-propoxy-N-oxopiperidine, 2,6-dimethyl-4-butoxy-N-oxopiperidine, 2,6-dimethyl-4-pentoxy-N-oxopiperidine, 2,6-dimethyl-4-hexyloxy-N-oxopiperidine, 2,2,6,6-tetramethyl-4-methoxy-N-oxopiperidine, 2,2,6,6-tetramethyl-4-hexoxy-N-oxopiperidine, 2,2,6,6-tetramethyl-4-propoxy-N-oxopiperidine, 2,2,6,6-tetramethyl-4-butoxy-N-oxopiperidine, 2,2,6,6-tetramethyl-4-pentoxy-N-oxopiperidine, 2,2,6,6-tetramethyl-4-hexyloxy-N-oxopiperidine, 2,6-dimethyl-4-cyano-N-oxopiperidine, 2,2,6,6-tetramethyl-4-cyano-N-oxopiperidine, 2,6-dimethyl-4-O-formyl-N-oxopiperidine, 2,6-dimethyl-4-O-acetyl-N-oxopiperidine, 2,6-dimethyl-4-O-propionyl-N-oxopiperidine, 2,6-dimethyl-4-O-butyryl-N-oxopiperidine, 2,6-dimethyl-4-O-benzoyl-N-oxopiperidine, 2,6-dimethyl-4-O-phenylacetyl-N-oxopiperidine, 2.6-dimethyl-4-O-n-butenoyl-N-oxopiperidine, 2,6-dimethyl-4-O-iso-butenoyl-N-oxopiperidine, 2,2,6,6-tetramethyl-4-cyano-N-oxopiperidine, 2,2,6,6-tetramethyl-4-formyl-N-oxopiperidine, 2,2,6,6-tetramethyl-4-O-acetyl-N-oxopiperidine, 2,2,6,6-tetramethyl-4-O-propionyl-N-oxopiperidine, 2,2,6,6-tetramethyl-4-O-butyryl-N-oxopiperidine, 2,2,6,6-tetramethyl-4-O-benzoyl-N-oxopiperidine, 2,2,6,6-tetramethyl-4-O-phenylacetyl-N-oxopiperidine, 2,2,6,6-tetramethyl-4-O-(3-butenoyl)-N-oxopiperidine, 2,2,6,6-tetramethyl-4-(2-butenoyl)-N-oxopiperidine, 2,6-dimethyl-4-carboxamido-N-oxopiperidine, 2,6-dimethyl-4-acetamido-N-oxopiperidine, 2,2,6,6-tetramethyl-4-carboxamido-N-oxopiperidine, 2,2,6,6-tetramethyl-4-acetamido-N-oxopiperidine, 2,6-dimethyl-4-amino-N-oxopiperidine, 2,2,6,6-tetramethyl-4-amino-N-oxopiperidine, 2,6-dimethyl-4-maleimide-N-oxopiperidine, 2,2,6,6-tetramethyl-4-maleimide-N-oxopiperidine, 2-fluoro-N-oxopiperidine, 3-fluoro-N-oxopiperidine, 4-fluoro-N-oxopiperidine, 2,3-difluoro-N-oxopiperidine, 2,4-difluoro-N-oxopiperidine, 2,5-difluoro-N-oxopiperidine, 2,6-difluoro-N-oxopiperidine, 2-bromo-N-oxopiperidine, 3-bromo-N-oxopiperidine, 4-bromo-N-oxopiperidine, 2,3-dibromo-N-oxopiperidine, 2,4-dibromo-N-oxopiperidine, 2,5-dibromo-N-oxopiperidine, 2,6-dibromo-N-oxopiperidine, or a compound represented by the following formula:

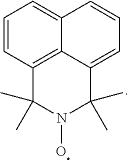

In addition, in some example preferred embodiments of the present disclosure, the electrolyte additive may include one of the following compounds or any combination thereof:

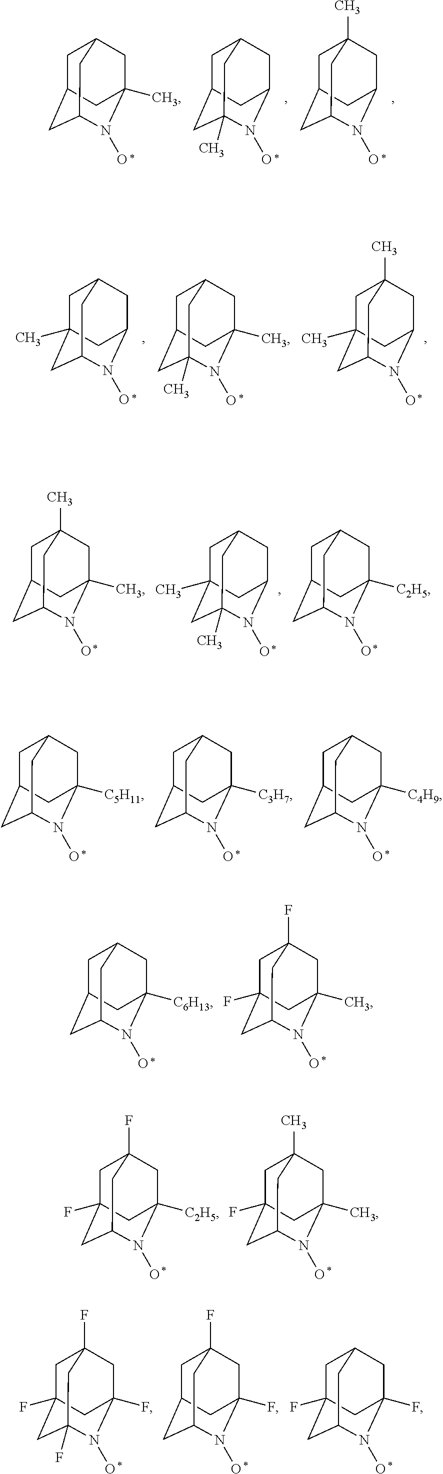

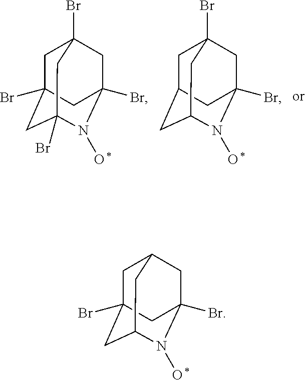

In other example preferred embodiments, the electrolyte additive may include one of the following compounds or any combination thereof:

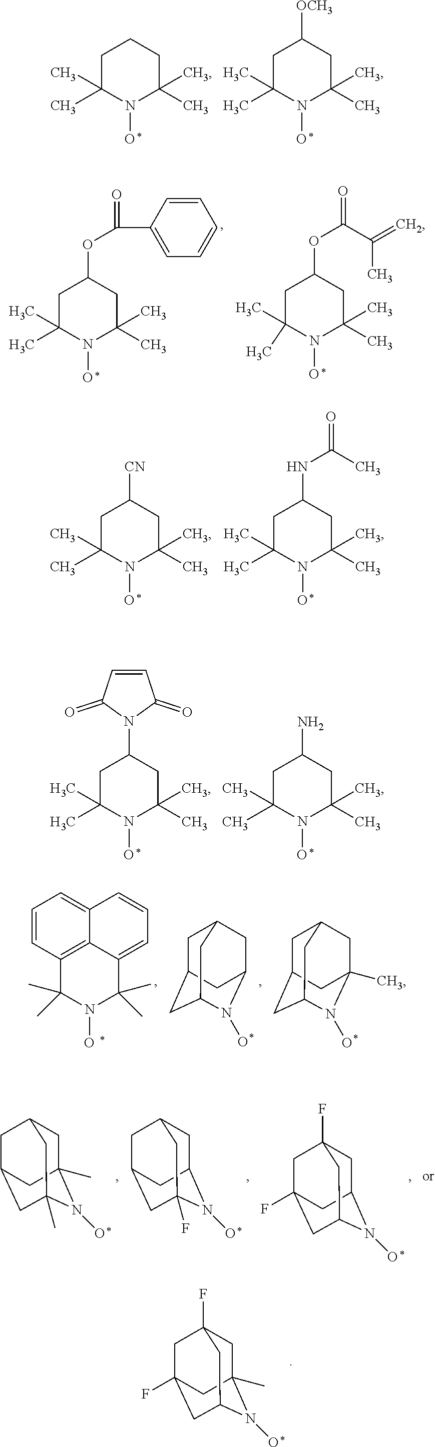

In another example preferred embodiment of the present disclosure, an electrolyte includes an organic solvent, a lithium salt, a film-forming additive, and the electrolyte additive as described above. In an example preferred embodiment, the film-forming additive in the electrolyte of the present disclosure includes fluoroethylene carbonate and a derivative thereof, and vinylene carbonate and a derivative thereof. Because the electrolyte additive according to an example preferred embodiment of the present disclosure is included, the electrolyte according to an example preferred embodiment of the present disclosure may effectively form an SEI film on the surface of a negative electrode during the first charging and discharging cycle process of the battery, such that the decomposition of the solvent is inhibited. In addition, because the electrolyte includes both the film-forming additive and the electrolyte additive according to an example preferred embodiment of the present disclosure, the resistance of the lithium ion secondary battery during the first film-forming process and a usage amount of the film-forming additive used in the electrolyte may be significantly reduced.

In some example preferred embodiments of the present disclosure, in the electrolyte according to an example preferred embodiment of the present disclosure, an amount of the electrolyte additive ranges of about 0.01 parts by weight to about 1 part by weight, based on 100 parts by weight of a total weight of the organic solvent, the lithium salt, and the film-forming additive. The compound containing the N—O. free radical according to an example preferred embodiment of the present disclosure is a reversible redox material, and it may reversibly carry out a redox reaction in the electrolyte of the lithium ion secondary battery, namely, during a charging and discharging cycle process, the compound containing the N—O. free radical according to an example preferred embodiment of the present disclosure only plays a role similar to a catalyst, it may not be completely consumed, so a small addition amount may play a role.

When the addition amount of the electrolyte additive according to an example preferred embodiment of present disclosure is in the abovementioned range, the electrolyte additive may promote the film-forming additive to effectively form a solid electrolyte membrane. In addition, the electrolyte additive within this range may effectively increase the decomposition potential of fluoroethylene carbonate, and the FEC decomposed at the high potential level is more conducive to form the stable SEI film.

While the amount of the electrolyte additive according to an example preferred embodiment of present disclosure is less than about 0.01 parts by weight, the electrolyte additive in the electrolyte is insufficient to effectively increase the decomposition potential of the FEC, and therefore the technical effects described above may not be achieved sufficiently. While the amount of the electrolyte additive according to an example preferred embodiment of present disclosure is higher than about 1 part by weight, the amount of the electrolyte additive in the electrolyte is excessive, although the dissolution of transition metal may be better inhibited, however, the thickness of membrane formed on the negative electrode is oversized, so that the battery resistance is increased, thereby the cycle characteristics are decreased.

In various example preferred embodiments of the present disclosure, according to different combinations of the lithium salt and organic solvent, a minimum value of the amount of the electrolyte additive of present disclosure in the electrolyte should be greater than about 0.01 parts by weight, about 0.02 parts by weight, about 0.03 parts by weight, about 0.04 parts by weight, about 0.05 parts by weight, about 0.06 parts by weight, about 0.07 parts by weight, about 0.08 parts by weight, about 0.09 parts by weight, about 0.1 parts by weight, about 0.11 parts by weight, about 0.12 parts by weight, about 0.13 parts by weight, about 0.15 parts by weight, about 0.16 parts by weight, about 0.17 parts by weight, about 0.18 parts by weight or about 0.19 parts by weight, based on 100 parts by weight of the total weight of the organic solvent, lithium salt and film-forming additive. In addition, according to the different combinations of the organic solvent, lithium salt and film-forming additive, a maximum value of the amount of the electrolyte additive of present disclosure in the electrolyte should be less than about 1 part by weight, about 0.9 parts by weight, about 0.8 parts by weight, about 0.7 parts by weight, about 0.6 parts by weight, about 0.5 parts by weight, about 0.49 parts by weight, about 0.48 parts by weight, about 0.47 parts by weight, about 0.46 parts by weight, about 0.45 parts by weight, about 0.44 parts by weight, about 0.43 parts by weight, about 0.42 parts by weight, about 0.41 parts by weight, about 0.4 parts by weight, about 0.35 parts by weight, about 0.3 parts by weight, about 0.25 parts by weight or about 0.2 parts by weight, based on 100 parts by weight of the total weight of the organic solvent, lithium salt and film-forming additive.

Specifically, the amount of the electrolyte additive according to an example preferred embodiment of present disclosure in the electrolyte may be within the following range: from about 0.01 parts by weight to about 1 part by weight, from about 0.02 parts by weight to about 0.9 parts by weight, from about 0.03 parts by weight to about 0.8 parts by weight, from about 0.04 parts by weight to about 0.7 parts by weight, from about 0.05 parts by weight to about 0.6 parts by weight, from about 0.06 parts by weight to about 0.5 parts by weight, from about 0.07 parts by weight to about 0.4 parts by weight, from about 0.08 parts by weight to about 0.3 parts by weight, from about 0.09 parts by weight to about 0.2 parts by weight, from about 0.01 parts by weight to about 0.9 parts by weight, from about 0.01 parts by weight to about 0.8 parts by weight, from about 0.01 parts by weight to about 0.7 parts by weight, from about 0.01 parts by weight to about 0.6 parts by weight, from about 0.01 parts by weight to about 0.5 parts by weight, from about 0.05 parts by weight to about 0.46 parts by weight, from about 0.06 parts by weight to about 0.45 parts by weight, from about 0.07 parts by weight to about 0.44 parts by weight, from about 0.08 parts by weight to about 0.43 parts by weight, from about 0.09 parts by weight to about 0.42 parts by weight, from about 0.1 parts by weight to about 0.41 parts by weight, from about 0.11 parts by weight to about 0.4 parts by weight, from about 0.12 parts by weight to about 0.35 parts by weight, from about 0.13 parts by weight to about 0.3 parts by weight, from about 0.14 parts by weight to about 0.25 parts by weight, from about 0.15 parts by weight to about 0.2 parts by weight, about 0.01 parts by weight to about 0.2 parts by weight, from about 0.02 parts by weight to about 0.2 parts by weight parts by weight, from about 0.15 parts by weight to about 0.5 parts by weight, from about 0.13 parts by weight to about 0.5 parts by weight, from about 0.12 parts by weight to about 0.25 parts by weight, from about 0.01 parts by weight to about 0.25 parts by weight, or from about 0.01 parts by weight to about 0.35 parts by weight, based on 100 parts by weight of the total weight of the organic solvent, lithium salt and film-forming additive.

In example preferred embodiments of the present disclosure, the organic solvent of the non-aqueous electrolyte may be any non-aqueous solvents which are used for non-aqueous electrolyte solution so far. Instances include but not limited to: linear or cyclic carbonates, such as ethylene carbonate, propylene carbonate, butylene carbonate, diethyl carbonate, dimethyl carbonate, ethyl methyl carbonate, dipropyl carbonate, and fluoroethylene carbonate; ethers, such as 1,2-dimethoxyethane, 1,2-diethoxyethane, gamma-butyrolactone, tetrahydrofuran, 2-methyltetrahydrofuran, 1,3-dioxolane, 4-methyl-1,3-dioxolane, and diethyl ether; sulfones, such as sulfolane, and methyl sulfolane; nitriles, such as acetonitrile, propionitrile, and arylonitrile; and esters, such as acetates, propionates, and butyrates, and the like. These non-aqueous solvents may be separately used or at least two solvents are combined to be used. In some embodiments of the present disclosure, a preferable electrolyte comprises ethylene carbonate, propylene carbonate, butylene carbonate, fluoroethylene carbonate, diethyl carbonate, dipropyl carbonate, ethyl methyl carbonate, carbonic acid ethylene ester and/or dimethyl carbonate, or any combination thereof. In a preferable embodiment, at least one carbonic ester is used as the organic solvent of the electrolyte of the present disclosure. In some other preferable embodiments, the above non-aqueous solvents may be arbitrarily used and combined so as to form the electrolyte solution in accordance with different requirements.

In example preferred embodiments of the present disclosure, no special limitation for a lithium salt component contained in the electrolyte, and the known lithium salt in prior art which may be used for a lithium battery electrolyte may be adopted. The examples of the lithium salt include but not limited to LiCl, LiBr, LiPF₆, LiBF₄, LiAsF₆, LiClO₄, LiB(C₆H₅)₄, LiCH₃SO₃, LiCF₃SO₃, LiN(SO₂F)₂, LiN(SO₂CF₃)₂, LiC(SO₂CF₃)₃, LiAlCl₄ and/or LiSiF₆, or any combination thereof.

In another example preferred embodiment of the present disclosure, a lithium ion secondary battery is provided, and the lithium ion secondary battery includes: a positive electrode, a negative electrode, a separator, and the electrolyte as described above. Because the lithium ion secondary battery according to an example preferred embodiment of the present disclosure uses the electrolyte as described above, the lithium ion secondary battery has excellent electric performance at high-temperature and high-voltage.

The positive electrode according to an example preferred embodiment of the present disclosure includes a positive electrode current collector and a positive electrode active substance layer containing a positive electrode active substance. The positive electrode active substance layer is formed on two surfaces of the positive electrode current collector. Metal foil, such as aluminum foil, nickel foil and stainless steel foil, may be used as the positive electrode current collector.

The positive electrode active substance layer includes one, two or more of positive electrode materials which are used as the positive electrode active substance and are capable of absorbing and releasing lithium ions, and if necessary, other materials may be contained, for example a positive electrode binder and/or a positive electrode conductive agent.

Preferably, the positive electrode material is a lithium-containing compound. Instances of the lithium-containing compound include a lithium-transition metal composite oxide, a lithium-transition metal phosphate compound, and the like. The lithium-transition metal composite oxide is an oxide containing Li and one, or two or more of the transition metals which are used as composition elements, and the lithium-transition metal phosphate compound is a phosphate compound containing Li and one, or two or more of transition metal which are used as the composition elements. In such compounds, the transition metal is advantageously any one, or two or more of Co, Ni, Mn, Fe, and the like.

Instances of the lithium-transition metal composite oxide include LiCoCg, LiNiCg, and the like. Instances of the lithium-transition metal phosphate compound include, for example LiFePO₄, LiFe₁₋ᵤMnᵤPO₄ (0<u<1), and the like.

In some example preferred embodiments of the present disclosure, the positive electrode material may be a ternary positive electrode material, such as lithium nickel cobalt aluminate (NCA) or lithium nickel cobalt manganate (NCM). Specific examples may be NCA, LiₓNi_{y}Co_{z}Al_{1-y-z}O₂ (1≤x≤1.2, 0.5≤y≤1, and 0≤z≤0.5). NCM, LiNiₓCo_{y}Mn_{z}O₂ (x+y+z=1, 0<x<1, 0<y<1, 0<z<1). Specific examples of the positive electrode materials may include, but are not limited to, the following materials: LiNiO₂, LiCoO₂, LiCo_{0.98}Al_{0.01}Mg_{0.01}O₂, LiNi_{0.5}Co_{0.2}Mn_{0.3}O₂, LiNi_{0.8}Co_{0.15}Al_{0.05}O₂, LiNi_{0.33}Co_{0.33}Mn_{0.33}O₂, Li_{1.2}Mn_{0.52}Co_{0.175}Ni_{0.1}O₂ and Li_{1.15} (Mn_{0.65}Ni_{0.22}Co_{0.13})O₂, LiFePO₄, LiMnPO₄, LiFe_{0.5}Mn_{0.5}PO₄ and LiFe_{0.3}Mn_{0.7}PO₄.

In addition, the positive electrode material may be, for example any one, or two or more of an oxide, a disulfide, a chalcogen compound, a conductive polymer, and the like. Instances of the oxide include, for example a titanium oxide, a vanadium oxide, manganese dioxide, and the like. Instances of the disulfide include, for example titanium disulfide, molybdenum sulfide, and the like. Instances of the chalcogen compound include, for example niobium selenide and the like. Instances of the conductive polymer include, for example sulfur, polyaniline, polythiophene, and the like. However, the positive electrode material may be a material different from those mentioned above.

An instance of the positive electrode conductive agent includes a carbon material, for example graphite, carbon black, acetylene black, and Ketjen black. These may be independently used, or two or more of them may be mixed for using. It is to be noted that the positive electrode conductive agent may be a metal material, a conductive polymer, or an analogue, only if it has electrical conductivity.

Examples of the positive electrode binder include synthetic rubber and a polymer material. For example, the synthetic rubber may be styrene-butadiene rubber, fluororubber and ethylene-propylene-diene rubber. For example, the polymer material may be polyvinylidene fluoride, polyvinyl alcohol, carboxymethyl cellulose (CMC), starch, hydroxypropyl cellulose, regenerated cellulose, polyvinylpyrrolidone, tetrafluoroethylene, polyethylene, polypropylene, lithium polyacrylate, ethylene-propylene-diene terpolymer (EPDM), sulfonated EPDM and polyimide. These may be independently used, or two or more of them may be mixed for using.

The negative electrode according to an example preferred embodiment of the present disclosure includes a negative electrode current collector and a negative electrode active substance layer containing a negative electrode active substance. The negative electrode active substance layer is formed on two surfaces of the negative electrode current collector. A metal foil, such as a copper (Cu) foil, a nickel foil, or a stainless steel foil, may be used as the negative electrode current collector.

The negative electrode active substance layer contains a material which is used as the negative electrode active substance and is capable of absorbing and releasing lithium ions, and may contain another material if necessary, for example a negative electrode binder and/or a negative electrode conductive agent. Details of the negative electrode binder and the negative electrode conductive agent are the same as that of the positive electrode binder and the positive electrode conductive agent for example.

The active material of the negative electrode is selected from any one or any combination of lithium metal, lithium alloy, carbon material, silicon or tin and oxides thereof.

Because the carbonaceous material has a low electric potential when lithium ions are absorbed, high energy density may be achieved, and battery capacity may be increased. Furthermore, the carbonaceous material also acts as the conductive agent. This type of the carbonaceous material is a material or an analogue obtained by coating a natural graphite and/or an artificial graphite, for example, with amorphous carbon. It is to be noted that a shape of the carbonaceous material is a fiber form, a spherical shape, a granular form, a flake form, or a similar shape. Silicon-based materials include nano-silicon, silicon alloys, and silicon-carbon composite materials composed of SiO_{w} and graphite. Preferably, the SiO_{w} is SiOₓ(1<x<2), silicon oxide or other silicon-based materials.

Besides, the negative electrode material may be one, or two or more of easy-graphited carbon, difficult-graphited carbon, a metallic oxide, a polymer compound, and the like. Instances of the metallic oxide include an iron oxide, a ruthenium oxide, a molybdenum oxide, and the like. Instances of the polymer compound include polyacetylene, polyaniline, polypyrrole, and the like. However, the negative electrode material may be another material different from those as described above.

The separator according to an example preferred embodiment of the present disclosure is used for separating the positive electrode and the negative electrode in the battery, and enabling ions to pass through, at the same time preventing current short circuit caused by contact between the two electrode pieces. The separator may be, for example, a porous membrane formed by synthetic resin, ceramic, or similar substances, and a laminating membrane laminated by two or more porous membranes. Instances of the synthetic resin include for example polytetrafluoroethylene, polypropylene, polyethylene, cellulose, and the like.

In an example preferred embodiment of the present disclosure, when charging is performed, for example, lithium ions are released from the positive electrode and absorbed in the negative electrode through the non-aqueous electrolyte dipping in the separator. While discharging is performed, for example, the lithium ions are released from the negative electrode and absorbed in the positive electrode through the non-aqueous electrolyte impregnating with the separator.

In another example preferred embodiment of the present disclosure, use of an electrolyte additive according to an example preferred embodiment of the present disclosure in preparation of a lithium ion secondary battery is provided. After the electrolyte additive according to an example preferred embodiment of the present disclosure is added to the lithium ion secondary battery, during the first charging and discharging cycle process, the electrolyte additive according to an example preferred embodiment of the present disclosure is preferentially decomposed to generate electrons, and the electrons may promote the film-forming additive in the electrolyte (preferably FEC) to be quickly decomposed and form a membrane on the negative electrode, so as to provide a stable SEI film. Preferably, in some example preferred embodiments of the present disclosure, the electrolyte additive of the present disclosure participates in the film formation of the film-forming additive, thereby a SEI film of a mixture is formed with the film-forming additive on the surface of the negative electrode.

Example preferred embodiments of the present disclosure are further described in detail in combination with specific examples below, these examples may not be understood to limit a scope of protection claimed by the present disclosure.

### Examples of Lithium Nickel Cobalt Aluminate Battery

### Preparation of Electrolyte

### Example 1

20 g of ethylene carbonate, 62 g of dimethyl carbonate are mixed with 18 g of lithium hexafluorophosphate to prepare a basic electrolyte, 1 g of fluoroethylene carbonate (FEC) and 0.01 g of electrolyte additive AZADO are added into the basic electrolyte. After uniformly stirring, it is used for standby application. Wherein the AZADO is a compound shown in the following formula:

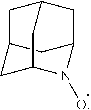

### Example 2

20 g of ethylene carbonate, 62 g of dimethyl carbonate are mixed with 18 g of lithium hexafluorophosphate to prepare a basic electrolyte, 1 g of fluoroethylene carbonate (FEC) and 0.1 g of electrolyte additive AZADO are added into the basic electrolyte. After uniformly stirring, it is used for standby application.

### Example 3

20 g of ethylene carbonate, 62 g of dimethyl carbonate are mixed with 18 g of lithium hexafluorophosphate to prepare a basic electrolyte, 1 g of fluoroethylene carbonate (FEC) and 0.5 g of electrolyte additive AZADO are added into the basic electrolyte. After uniformly stirring, it is used for standby application.

### Example 4

20 g of ethylene carbonate, 62 g of dimethyl carbonate are mixed with 18 g of lithium hexafluorophosphate to prepare a basic electrolyte, 1 g of fluoroethylene carbonate (FEC) and 0.01 g of electrolyte additive CN-TEMPO are added into the basic electrolyte. After uniformly stirring, it is used for standby application. Wherein the CN-TEMPO is a compound shown in the following formula:

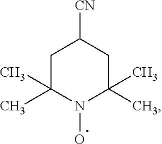

namely 2,2,6,6-tetramethyl-4-cyano-N-oxopiperidine.

### Example 5

20 g of ethylene carbonate, 62 g of dimethyl carbonate are mixed with 18 g of lithium hexafluorophosphate to prepare a basic electrolyte. 1 g of fluoroethylene carbonate (FEC) and 0.1 g of electrolyte additive CN-TEMPO are added into the basic electrolyte. After uniformly stirring, it is used for standby application.

### Example 6

20 g of ethylene carbonate, 62 g of dimethyl carbonate are mixed with 18 g of lithium hexafluorophosphate to prepare a basic electrolyte. 1 g of fluoroethylene carbonate (FEC) and 0.5 g of electrolyte additive CN-TEMPO are added into the basic electrolyte. After uniformly stirring, it is used for standby application.

### Example 7

20 g of ethylene carbonate, 62 g of dimethyl carbonate are mixed with 18 g of lithium hexafluorophosphate to prepare a basic electrolyte. 1 g of fluoroethylene carbonate (FEC) and 0.01 g of electrolyte additive TEMPO are added into the basic electrolyte. After uniformly stirring, it is used for standby application. Wherein the TEMPO is a compound shown in the following formula:

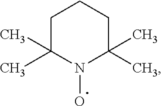

namely 2,2,6,6-tetramethyl-N-oxopiperidine.

### Example 8

20 g of ethylene carbonate, 62 g of dimethyl carbonate are mixed with 18 g of lithium hexafluorophosphate to prepare a basic electrolyte. 1 g of fluoroethylene carbonate (FEC) and 0.1 g of electrolyte additive TEMPO are added into the basic electrolyte. After uniformly stirring, it is used for standby application.

### Example 9

20 g of ethylene carbonate, 62 g of dimethyl carbonate are mixed with 18 g of lithium hexafluorophosphate to prepare a basic electrolyte. 1 g of fluoroethylene carbonate (FEC) and 0.5 g of electrolyte additive TEMPO are added into the basic electrolyte. After uniformly stirring, it is used for standby application.

### Comparative Example 1

20 g of ethylene carbonate, 62 g of dimethyl carbonate are mixed with 18 g of lithium hexafluorophosphate to prepare a basic electrolyte. 1 g of fluoroethylene carbonate (FEC) is added into the basic electrolyte. After uniformly stirring, it is used for standby application.

### Comparative Example 2

20 g of ethylene carbonate, 62 g of dimethyl carbonate are mixed with 18 g of lithium hexafluorophosphate to prepare a basic electrolyte. 8 g of fluoroethylene carbonate (FEC) is added into the basic electrolyte. After uniformly stirring, it is used for standby application.

### Comparative Example 3

20 g of ethylene carbonate, 62 g of dimethyl carbonate are mixed with 18 g of lithium hexafluorophosphate to prepare a basic electrolyte. 1 g of fluoroethylene carbonate (FEC) and 3 g of electrolyte additive AZADO are added into the basic electrolyte. After uniformly stirring, it is used for standby application.

### Comparative Example 4

20 g of ethylene carbonate, 62 g of dimethyl carbonate are mixed with 18 g of lithium hexafluorophosphate to prepare a basic electrolyte. 1 g of fluoroethylene carbonate (FEC) and 3 g of electrolyte additive CN-TEMPO are added into the basic electrolyte. After uniformly stirring, it is used for standby application.

### Comparative Example 5

20 g of ethylene carbonate, 62 g of dimethyl carbonate are mixed with 18 g of lithium hexafluorophosphate so as to prepare a basic electrolyte. 1 g of fluoroethylene carbonate (FEC) and 3 g of electrolyte additive TEMPO are added into the basic electrolyte. After uniformly stirring, it is used for standby application.

### Preparation of Battery

### Example 10

### Preparation of Positive Electrode

95.5 g of lithium nickel cobalt aluminate NCA as positive electrode active material, 2.5 g of conductive carbon black, 1.9 g of polyvinylidene fluoride and 0.1 g of polyvinylpyrrolidone as dispersant are mixed to obtain a positive electrode mixture, and the obtained positive electrode mixture is dispersed in N-methylpyrrolidone to obtain positive electrode mixture slurry. After that, an aluminum foil is coated by the positive electrode mixture slurry to obtain a positive electrode current collector. The positive electrode current collector is dried, and a positive electrode piece is formed by using a punch-forming process.

### Preparation of Negative Electrode

95.85 g of mixture of silicon oxide (SiOₓ, 1<x<2) and graphite powder, 1 g of Super-P as conductive agent, 3.15 g of CMC (sodium carboxymethyl cellulose) and SBR (styrene butadiene rubber) as binder are added into an appropriate amount of water to prepare negative electrode slurry. Then, a copper foil is coated by the obtained negative electrode slurry uniformly to obtain a negative electrode current collector. The negative electrode current collector is dried and a negative electrode piece is formed by a punch-forming process.

### Assembly of Battery

A CR2016 button battery is assembled in a dry laboratory. The positive electrode piece obtained in the above steps is used as a positive electrode, the negative electrode piece obtained in the above steps is used as a negative electrode, and the electrolyte prepared in Example 1 is used as electrolyte. The positive electrode, the negative electrode and the separator are assembled with a battery case of the button battery. After being assembled, the battery rests for 24 h to be aged, thereby a NCA button battery is obtained.

### Example 11

A button battery is prepared similarly to Example 10, and a difference is that the electrolyte prepared in Example 2 is used as electrolyte of the button battery prepared in Example 11.

### Example 12

A button battery is prepared similarly to Example 10, and a difference is that the electrolyte prepared in Example 3 is used as electrolyte of the button battery prepared in Example 12.

### Example 13

A button battery is prepared similarly to Example 10, and a difference is that the electrolyte prepared in Example 4 is used as electrolyte of the button battery prepared in Example 13.

### Example 14

A button battery is prepared similarly to Example 10, and a difference is that the electrolyte prepared in Example 5 is used as electrolyte of the button battery prepared in Example 14.

### Example 15

A button battery is prepared similarly to Example 10, and a difference is that the electrolyte prepared in Example 6 is used as electrolyte of the button battery prepared in Example 15.

### Example 16

A button battery is prepared similarly to Example 10, and a difference is that the electrolyte prepared in Example 7 is used as electrolyte of the button battery prepared in Example 16.

### Example 17

A button battery is prepared similarly to Example 10, and a difference is that the electrolyte prepared in Example 8 is used as electrolyte of the button battery prepared in Example 17.

### Example 18

A button battery is prepared similarly to Example 10, and a difference is that the electrolyte prepared in Example 9 is used as electrolyte of the button battery prepared in Example 18.

### Comparative Example 6

A button battery is prepared similarly to Example 10, and a difference is that the electrolyte prepared in Comparative Example 1 is used as electrolyte of the button battery prepared in Comparative Example 6.

### Comparative Example 7

A button battery is prepared similarly to Example 10, and a difference is that the electrolyte prepared in Comparative Example 2 is used as electrolyte of the button battery prepared in Comparative Example 7.

### Comparative Example 8

A button battery is prepared similarly to Example 10, and a difference is that the electrolyte prepared in Comparative Example 3 is used as electrolyte of the button battery prepared in Comparative Example 8.

### Comparative Example 9

A button battery is prepared similarly to Example 10, and a difference is that the electrolyte prepared in Comparative Example 4 is used as electrolyte of the button battery prepared in Comparative Example 9.

### Comparative Example 10

A button battery is prepared similarly to Example 10, and a difference is that the electrolyte prepared in Comparative Example 5 is used as electrolyte of the button battery prepared in Comparative Example 10.

### Test of Battery Performance

At a room temperature, the NCA button batteries of Examples 10-18 and Comparative Examples 6-10 are performed to charging-discharging test and resistance test at a voltage between 2.5 V and 4.45 V. Firstly, 0.1C cycle tests are performed on the batteries prepared in the above examples and comparative examples at 23° C. for 1 time, and then 0.5C charging and 5C discharging cycle tests are performed at 60° C. for 100 times, thereby a cycle retention rates of the batteries are determined. Finally, 0.5C charging tests are performed at 60° C. for 1 time to determine resistance values of the batteries. Experimental results are shown in Table 1 below.

TABLE 1
battery performance testing results

| | | Addition | | | | |
|---|---|---|---|---|---|---|
| | Type of | amount of | | Addition | | resistance after |
| | film- | film- | Type of | amount of | | charging and |
| | forming | forming | electrolyte | electrolyte | Cycle | discharging |
| | additive | additive | additive | additive | retention | cycle (Ω) |
| Example 10 | FEC | 1% | AZADO | 0.01% | 70.00% | 52 |
| Example 11 | FEC | 1% | AZADO | 0.1% | 72.20% | 41 |
| Example 12 | FEC | 1% | AZADO | 0.5% | 70.30% | 45 |
| Comparative | FEC | 1% | Not added | Not added | 69.70% | 53 |
| Example 6 | | | | | | |
| Comparative | FEC | 8% | Not added | Not added | 70.60% | 45 |
| Example 7 | | | | | | |
| Comparative | FEC | 1% | AZADO | 3% | 65% | 65 |
| Example 8 | | | | | | |
| Example 13 | FEC | 1% | CN-TEMPO | 0.01% | 69.80% | 52 |
| Example 14 | FEC | 1% | CN-TEMPO | 0.1% | 71.90% | 42 |
| Example 15 | FEC | 1% | CN-TEMPO | 0.5% | 70.10% | 46 |
| Comparative | FEC | 1% | CN-TEMPO | 3% | 64% | 69 |
| Example 9 | | | | | | |
| Example 16 | FEC | 1% | TEMPO | 0.01% | 69.7% | 52 |
| Example 17 | FEC | 1% | TEMPO | 0.1% | 71.30% | 42 |
| Example 18 | FEC | 1% | TEMPO | 0.5% | 70.10% | 47 |
| Comparative | FEC | 1% | TEMPO | 3% | 63% | 72 |
| Example 10 | | | | | | |

In Table 1, the “Addition amount of film-forming additive” and the “Addition amount of electrolyte additive” are both weight percentages based on a total weight of basic electrolyte.

It may be observed from the above testing results that the example preferred embodiments of the present disclosure achieve the following technical effects.

It may be observed from the experimental results that through comparing Examples 10-12 with Comparative Example 6, it may be seen that in the case of adding the electrolyte additive according to an example preferred embodiment of the present disclosure within the dosage range provided in example preferred embodiments of the present disclosure, the cycle retention rate of the battery is increased and the resistance after charging and discharging cycles is decreased. Compared with Comparative Example 7 in which the FEC is used only, it may be seen that in the case of the similar technical effects are achieved (namely, the cycle retention rate and the resistance after charging and discharging cycles are similar), the addition amount of the FEC in Comparative Example 7 is far greater than the addition amounts in Examples 10-12, therefore, in the case of using the electrolyte additive of the present disclosure, the use of the film-forming additive may be reduced significantly.

In addition, after comparing with Comparative Example 8, it may be seen that while the usage amount of the electrolyte additive according to an example preferred embodiment of an example preferred embodiment of the present disclosure exceeds a maximum value (for example, about 0.5%) of an example preferred embodiment of the present disclosure, the electrical performance of the battery may be adversely affected. After comparing Comparative Example 8 with Comparative Example 6, it may be seen that when usage amount of the electrolyte additive exceeds the range amount of an example preferred embodiment of the present disclosure, the cycle retention rate and resistance after charging and discharging cycles of the battery are degraded compared with not adding the electrolyte additive of the present disclosure.

### Other Examples

### Preparation of Electrolyte

### Example 19

42.6 g of ethylene carbonate and 42.6 g of propylene carbonate are mixed with 14 g of lithium hexafluorophosphate to prepare a basic electrolyte. 0.85 g of fluoroethylene carbonate (FEC) and 0.5 g of CN-TEMPO are added into the basic electrolyte. After uniformly stirring, it is used for standby application.

### Comparative Example 11

42.6 g of ethylene carbonate and 42.6 g of propylene carbonate are mixed with 14 g of lithium hexafluorophosphate to prepare a basic electrolyte. 0.85 g of fluoroethylene carbonate (FEC) is added into the basic electrolyte. After uniformly stirring, it is used for standby application.

### Preparation of Battery

### Example 20

### Preparation of Electrode Piece

80 g of silicon oxide (SiOₓ, 1<x<2), 10 g of conductive carbon black, 10 g of Li_{0.4}PAA (lithium polyacrylate) are added into an appropriate amount of water and stirring is performed to prepare slurry. Then, a copper foil is uniformly coated by the obtained slurry to obtain a current collector, and the current collector is dried to obtain an electrode piece.

### Assembly of Battery

A CR2016 button battery is assembled in a dry laboratory. The electrode piece produced in the above step is used as a positive electrode, lithium metal is used as a negative electrode is, and the electrolyte prepared in Example 19 is used as electrolyte. The positive electrode, the negative electrode, a separator and a battery case of the button battery are assembled. After being assembled, the battery rests for 24 h to be aged, thereby a silicon oxide-lithium half-cell button battery is obtained.

### Comparative Example 12

A silicon oxide-lithium half-cell button battery is prepared similarly to Example 20, a difference is that the electrolyte prepared in Comparative Example 11 is used as electrolyte of the half-cell button battery prepared in Comparative Example 12.

### Battery Performance Test

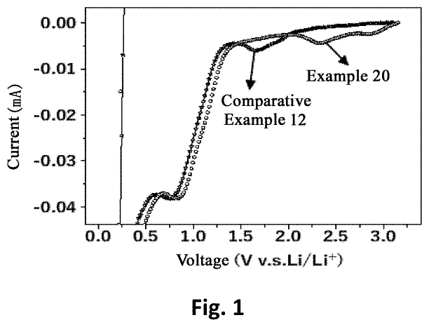

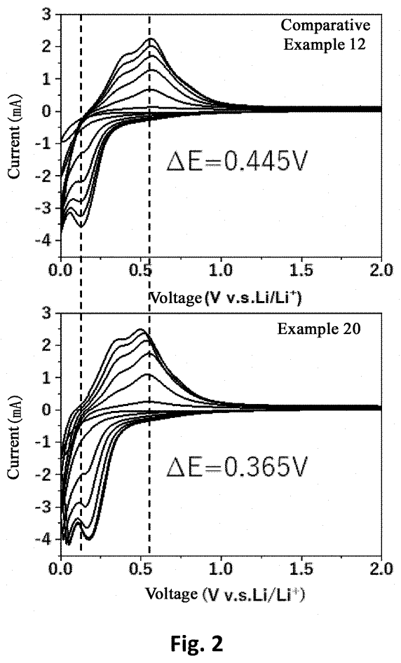

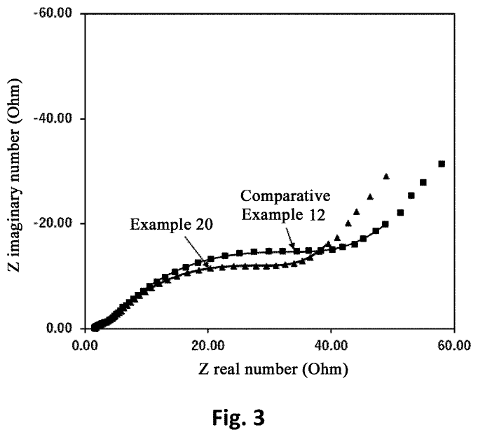

At a room temperature, charge-discharge test and resistance test are performed on the silicon oxide-lithium half-cell button batteries of Example 20 and Comparative Example 12 at a voltage between 0 and 1.5 V. Firstly, 0.05C charging and discharging cycle tests are performed on the batteries prepared in the above example and comparative example at 25° C. for one time, and then 0.5C charging tests are performed at 25° C. for one time, thereby resistance values of the batteries are determined. At the room temperature, a cyclic voltammetry test is performed on the silicon oxide-lithium half-cell button batteries of Example 20 and Comparative Example 12 at a voltage between 0 and 2V. The batteries in the above example and comparative example are firstly scanned at 25° C. from an open circuit voltage with a speed of 0.1 mV/s and scanned for 6 times, to obtain the cyclic voltammetry curves of the first cycle of the batteries, cyclic voltammetry curves and alternating current resistance spectrums of the batteries under full power. Experimental results are shown in FIGS. 1-3.

### Examples of Lithium Iron Phosphate Battery

### Preparation of Electrolyte

### Example 21

50 g of ethylene carbonate, 50 g of dimethyl carbonate are mixed with 16.4 g of lithium hexafluorophosphate to prepare a basic electrolyte. 1 g of fluoroethylene carbonate (FEC) and 1.174 g of electrolyte additive AZADO are added into the basic electrolyte. After uniformly stirring, it is used for standby application.

### Example 22

50 g of ethylene carbonate, 50 g of dimethyl carbonate are mixed with 16.4 g of lithium hexafluorophosphate to prepare a basic electrolyte. 1 g of fluoroethylene carbonate (FEC) and 1.174 g of electrolyte additive CN-TEMPO are added into the basic electrolyte. After uniformly stirring, it is used for standby application.

### Comparative Example 13

50 g of ethylene carbonate, 50 g of dimethyl carbonate are mixed with 16.4 g of lithium hexafluorophosphate to prepare a basic electrolyte. 1 g of fluoroethylene carbonate (FEC) is added into the basic electrolyte. After uniformly stirring, it is used for standby application.

### Preparation of Battery

### Example 23

### Preparation of Positive Electrode

93.4 g of lithium iron phosphate (LFP) of positive electrode active material, 2.5 g of conductive carbon black, 1.9 g of polyvinylidene fluoride and 0.1 g of dispersant polyvinylpyrrolidone are mixed to obtain a positive electrode mixture, and the obtained positive electrode mixture is dispersed in N-methylpyrrolidone to obtain positive electrode mixture slurry. After that, an aluminium foil is coated by the positive electrode mixture slurry to obtain a positive electrode current collector. The positive electrode current collector is dried, and a positive electrode piece is formed by using a punch-forming process.

### Preparation of Negative Electrode

80 g of silicon oxide (SiOₓ, 1<x<2), 10 g of conductive carbon black, 10 g of binder Li_{0.4}PAA are added into an appropriate amount of water and stirring is performed to prepare negative electrode slurry. After that, a copper foil is uniformly coated by the obtained negative electrode slurry to obtain a negative electrode current collector. The negative electrode current collector is dried, and a negative electrode piece is formed by using the punch-forming process.

### Assembly of Battery

A CR2016 button battery is assembled in a dry laboratory. The positive electrode piece obtained in the above step is used as a positive electrode, the negative electrode piece obtained in the above steps is used as a negative electrode, and the electrolyte prepared in Example 21 is used as electrolyte. The positive electrode, the negative electrode, a separator are assembled with a battery case of the button cell. After being assembled, the battery rests for 24 h to be aged, thereby a LFP button battery is obtained.

### Example 24

The button battery is prepared similarly to Example 23, a different is that the electrolyte prepared in Example 22 is used as electrolyte of the button battery prepared in Example 24.

### Comparative Example 14

The button battery is prepared similarly to Example 23, a different is that the electrolyte prepared in Comparative Example 13 is used as electrolyte of the button battery prepared in Comparative Example 14.

### Battery Performance Test

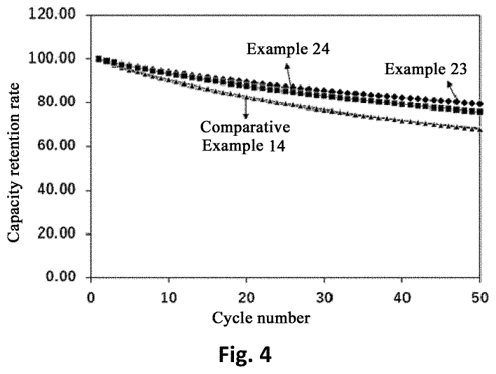

At a room temperature, a charging and discharging tests are performed on the LFP button batteries of Example 23, Example 24 and Comparative Example 14 at a voltage between 2.4 V and 3.75 V. Firstly, 0.1C cycle tests are performed on the batteries in the above examples and comparative example are at 25° C. for one time, and then 0.2C cycle tests are performed on for 50 times, thereby a cycle retention rate of the battery is determined. Experimental results are shown in FIG. 4.

The above descriptions are only example preferred embodiments of the present disclosure, and are not intend to limit the present disclosure, various changes and modifications may be made to the present disclosure by those skilled in the art. Within principles of the present disclosure, any modifications, equivalent replacements, improvements, and the like shall fall within the scope of protection of the present disclosure.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. An electrolyte additive comprising:
any one or more compounds in a group consisting of compounds according to Formula (1) and Formula (2): 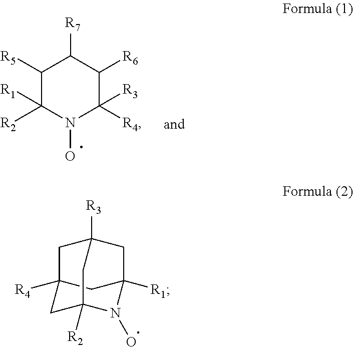
wherein
R₁ to R₄ are respectively independently selected from a group consisting of H, C₁₋₆ alkyl and halogen;
R₅ and R₆ are respectively independently selected from a group consisting of H, C₁₋₆ alkyl and aromatic hydrocarbon;
R₇ is independently selected from a group consisting of H, C₁₋₆ alkyl, C₁₋₆ alkoxy, a nitrile group, an ester group, an amide group, an amino group, and a maleimide group; and
optionally, R₅ and R₆ are respectively combined with R₇ or R₅ and R₆ are combined together with R₇ and atoms to which they are connected to form a 6-14-membered ring structure.

2. The electrolyte additive according to claim 1, wherein, in the compound of Formula (1), R₅ and R₆ are H respectively, and optionally, R₅ and R₆ are respectively combined with R₇ or R₅ and R₆ are combined together with R₇ and the atoms to which they are connected to form the 6-14-membered ring structure.

3. The electrolyte additive according to claim 1, wherein, in the compound of Formula (2), R₁ to R₄ are respectively independently selected from a group consisting of H, C₁₋₃ alkyl, and F.

4. The electrolyte additive according to claim 1, wherein the compound of Formula (1) is selected from the following compounds:

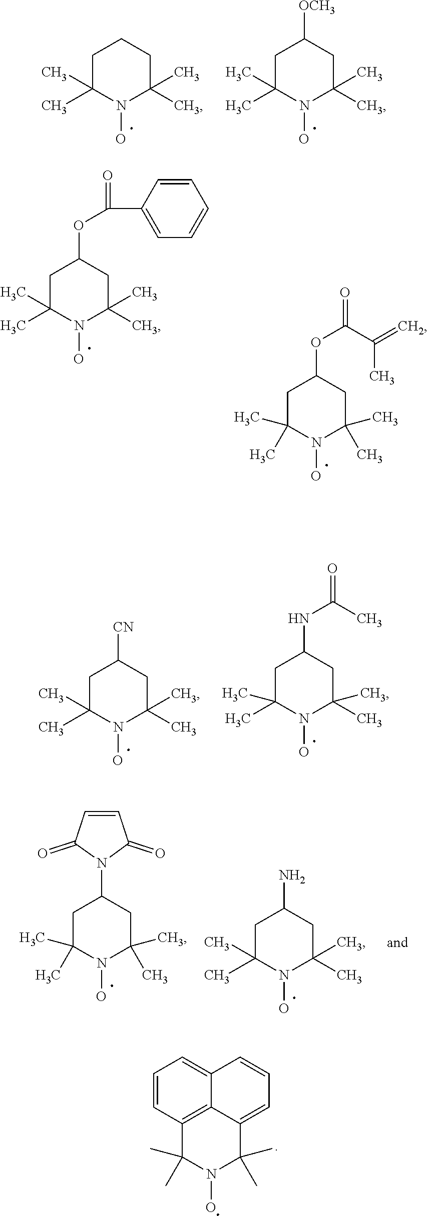

5. The electrolyte additive according to claim 1, wherein the compound of Formula (2) is selected from the following compounds:

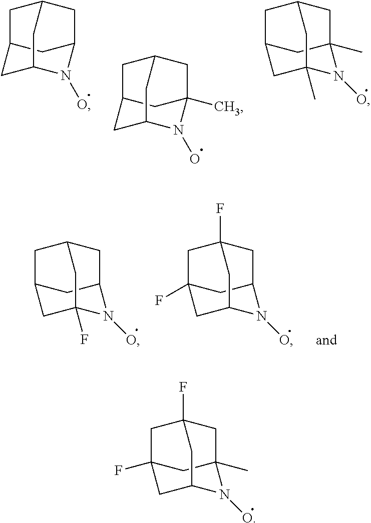

6. An electrolyte comprising an organic solvent, a lithium salt, a film-forming additive, and the electrolyte additive according to claim 1.

7. The electrolyte according to claim 6, wherein an amount of the electrolyte additive ranges from about 0.01 parts by weight to about 1 part by weight, based on 100 parts by weight of a total weight of the organic solvent, the lithium salt, and the film-forming additive.

8. The electrolyte according to claim 6, wherein, the lithium salt is selected from a group consisting of LiCl, LiBr, LiPF₆, LiBF₄, LiAsF₆, LiClO₄, LiB(C₆H₅)₄, LiCH₃SO₃, LiCF₃SO₃, LiN(SO₂F)₂, LiN(SO₂CF₃)₂, LiC(SO₂CF₃)₃, LiAlCl₄, LiSiF₆, or any combinations thereof.

9. A lithium ion secondary battery comprising:
a positive electrode;
a negative electrode;
a separator; and
the electrolyte according to claim 6.
